(19) United States
(12) Patent Application Publication
(10) Pub. No.: US 2018/0360641 A1
(43) Pub. Date: Dec. 20, 2018

(54) BACK-TRACTION DEVICE AND EXERCISE SYSTEM

(71) Applicant: Paul W. Budge, St. George, UT (US)

(72) Inventor: Paul W. Budge, St. George, UT (US)

(21) Appl. No.: 16/009,113

(22) Filed: Jun. 14, 2018

Related U.S. Application Data
(60) Provisional application No. 62/519,690, filed on Jun. 14, 2017.

(51) Int. Cl. A61F 5/048; A61H 1/02

(52) U.S. Cl. CPC A61F 5/048; A61H 1/0229; A61H 2203/0431; A61H 2201/1652; A61H 1/0292

(57) ABSTRACT

A spinal traction device is disclosed. In one embodiment, the device includes an elongated base having a first end and a second end, a first upright support member coupled to the first end and extending substantially perpendicularly from the base, the first upright support member comprising a first lifting mechanism and a first underarm support, and a second upright support member coupled to the second end and extending substantially perpendicularly from the base, the second upright support member comprising a second lifting mechanism and a second underarm support.

### CROSS-REFERENCES TO RELATED APPLICATIONS

This application claims the benefit of and claims priority to United States Provisional Patent Application No. 62/519690 entitled “BACK TRACTION DEVICE AND EXERCISE SYSTEM” and filed on Jun. 14, 2017 for Paul W. Budge, which is incorporated herein by reference.

### FIELD

This invention relates to traction devices generally and more particularly relates to a combination back-traction device and exercise system that incorporates a lifting mechanism to lift the upper body of a patient.

### BACKGROUND

Doctors and physical therapists have found that applying traction to patients suffering from back pain aids in recovery. Reducing compressive forces on the patient's spine (i.e., vertebrae, cartilage, etc.), and stretching the lumbar, sacral, or pelvic region is known to alleviate some kinds of back pain and provide therapeutic value. Horizontal immobilization (i.e., laying in bed) is effective, but inconvenient and often leads to boredom. Often patients are prone to ignore the order to remain in bed and attempt to return to normal activities, which may lead to further injury.

The lumbosacral spine (i.e., the base of the spine) supports large forces when a patient is sitting or standing. Traction devices may aid in reducing these forces. Generally, traction devices fall into one of two categories: traction devices that are used in bed, and traction devices that are used when sitting or standing (i.e., upright traction devices). Upright traction devices generally use a belt around the waist of the patient that bears some of the compressive forces of the upper body via supports that extend from the belt to some part of the upper body. For example, a second belt strapped around the chest of the patient, or crutch-like device that extends from the belt to the underarm area of the patient. Furthermore, traditional upright traction devices typically lack the capability of easily increasing or decreasing the amount of support.

### SUMMARY

A spinal traction device is disclosed. A system for a combination traction and exercise device is also disclosed. In one embodiment, the device includes an elongated base having a first end and a second end, a first upright support member coupled to the first end and extending substantially perpendicularly from the base, the first upright support member comprising a first lifting mechanism and a first underarm support, and a second upright support member coupled to the second end and extending substantially perpendicularly from the base, the second upright support member comprising a second lifting mechanism and a second underarm support.

The device may also include a chest harness configured to encircle a ribcage area of a patient, where the chest harness is coupled on a first side to the first underarm support member and coupled on a second side to the second underarm support member. In one embodiment, the elongated base comprises a first receiver, a second receiver, and a central bar disposed between, and slidable into, the first receiver and the second receiver, and configured to adjust a distance between the first upright support member and the second upright support member.

In one embodiment, the device includes a first planar pad coupled to the first receiver, and a second planar pad coupled to the second receiver. Each of the first planar pad and the second planar pad may comprise at least one curved edge. Additionally, each of the first planar pad and the second planar pad may have a width greater than a width of either the first upright support member or the second upright support member.

In one embodiment, the central bar is formed of a semi-rigid material. The device may also include an adjustable bracket disposed between the first or second underarm support member and the first or second side of the chest harness, where the adjustable bracket is configured to adjust a distance between a coupling point of the first or second side of the chest harness and a coupling point of the first or second underarm support member.

In certain embodiments, the device includes a controller configured to control the first lifting mechanism and the second lifting mechanism. The first lifting mechanism and the second lifting mechanism are selected from the group consisting of an infinitely adjustable spring, a pneumatic lifting strut, a hydraulic lifting strut, and an electronic lifting strut.

In one embodiment, the chest harness comprises at least one strap having a semi-rigid planar material disposed within a padded strap. Additionally, the chest harness may be formed of a pair of independently adjustable straps.

The system, in one embodiment, includes a support base comprising at least a pair of parallel supports and a center support, where the center support is disposed between and couples the parallel supports, the support base configured to engage a floor, and a pair of positioning braces, each of the pair of positioning braces coupled to one of the pair of parallel supports, where each of the pair of positioning braces comprises a plurality of locking positions. The system may also include a pair of support arms pivotally coupled to the support base and pivotable between a collapsed position an upright position, where one of the pair of support arms is coupled to one of the pair of parallel supports, and where one of the pair of positions braces is disposed between one of the pair of support arms and one of the pair of parallel supports.

In one embodiment, the system includes a pair of underarm support members, each of the pair of underarm support members coupled to one of the pair of support arms, and a chest harness configured to encircle a ribcage area of a patient, where the chest harness is coupled on a first side to one of the pair of underarm support members and is coupled on a second side to the other one of the pair of underarm support members. Each of the plurality of support arms, in one embodiment, comprises a locking pin configured to engage one of the plurality of locking positions. The plurality of locking positions trace an arc along which the locking pin travels when the pair of support arms pivots from the collapsed position to the upright position.

In one embodiment, the chest harness comprises at least one strap having a semi-rigid planar material disposed within a padded strap, and the chest harness comprises a pair of independently adjustable straps. The system may also include a cross brace coupling the pair of support members.

### BRIEF DESCRIPTION OF THE DRAWINGS

In order that the advantages of the subject matter may be more readily understood, a more particular description of the subject matter briefly described above will be rendered by reference to specific embodiments that are illustrated in the appended drawings. Understanding that these drawings depict only typical embodiments of the subject matter and are not therefore to be considered to be limiting of its scope, the subject matter will be described and explained with additional specificity and detail through the use of the drawings, in which:

FIG. 1 is a perspective view diagram of the spinal traction device in accordance with embodiments of the present disclosure;

FIG. 2 is another perspective view diagram illustrating one embodiment of the device in accordance with embodiments of the disclosure;

FIG. 3 is a perspective view diagram illustrating one embodiment of a patient using the device 100 in accordance with embodiments of the present disclosure;

FIGS. 4-8 are different view diagrams illustrating one embodiment of a framework that utilizes the chest harness and underarm supports in accordance with embodiments of the present disclosure; and

FIG. 9 is a partial cross-section diagram illustrating one embodiment of a ratcheting mechanism for locking a strut in a desired position in accordance with embodiments of the present disclosure.

### DETAILED DESCRIPTION

Described herein are embodiments of a spinal traction device that vastly improves on previous traction devices by allowing the full use of both arms and enabling full upper body mobility while a patient's back is being treated. As will be described in greater detail below, the spinal traction device includes a general U-shaped design with upright and extendable supports, to which are attached a padded chest harness and padded underarm supports. The spinal traction device, in some embodiments, lifts the upper body of the patient so that gravity and the mass of the patient pull down to decompress, straighten, and stretch the back. In other embodiments, also described below, the padded chest harness and padded underarm supports may be coupled to a framework that operates as a combination spinal traction device and exercise device.

Beneficially, the described embodiments disclose a spinal traction device that supports the weight of a patient in both the ribcage area and the underarm areas of the patient. This allows the patient to comfortably spend more time in the spinal traction device and increase the rehabilitating effect of decompressing and aligning the spine. Supporting the patient evenly around the ribcage and the underarm areas drastically reduces fatigue, and pain, and prevents blood flow restriction to the arms so the patent can comfortably use the disclosed device for longer periods of time (e.g., while watching a movie, or working at the office).

The description of elements in each figure may refer to elements of proceeding figures. Like numbers refer to like elements in all figures, including alternate embodiments of like elements.

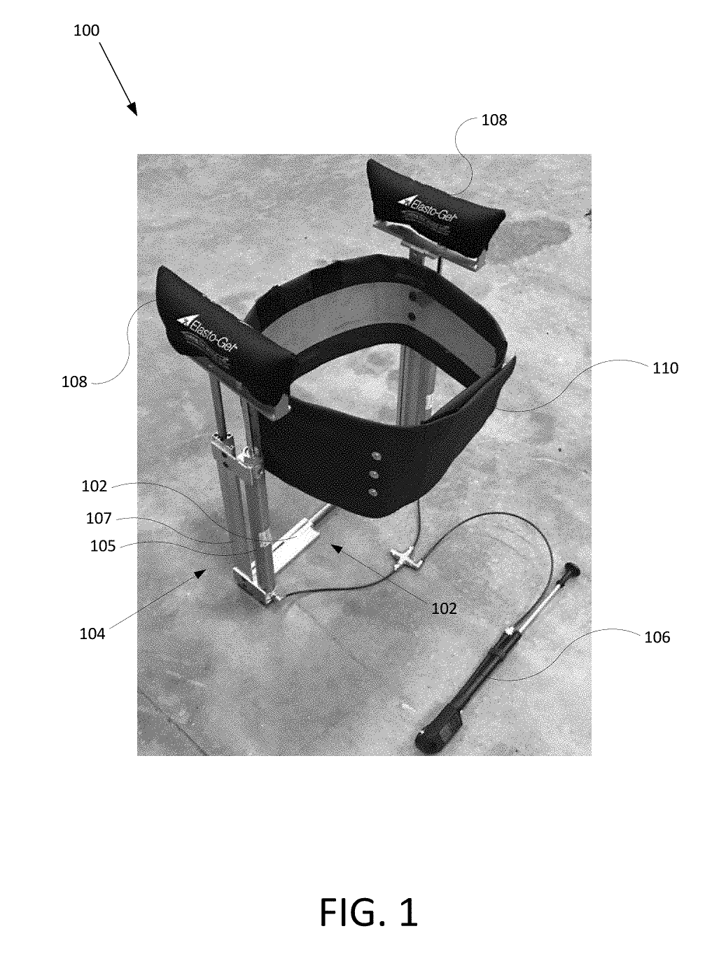

FIG. 1 is a perspective view diagram of the spinal traction device 100 (hereinafter “device 100”) in accordance with embodiments of the present disclosure. In on embodiment, the device 100 includes a base 102 that spaces apart, and extends between, at least a pair of upright supports 104. Each of the upright supports 104 extends upward perpendicularly from the base 102. The base 102 is configured to engage a seating surface (i.e., chair, bench, other seating surface, etc.), and the upright supports 104 extend upward from the base 102 to form a U-shape, or block U-shape as depicted.

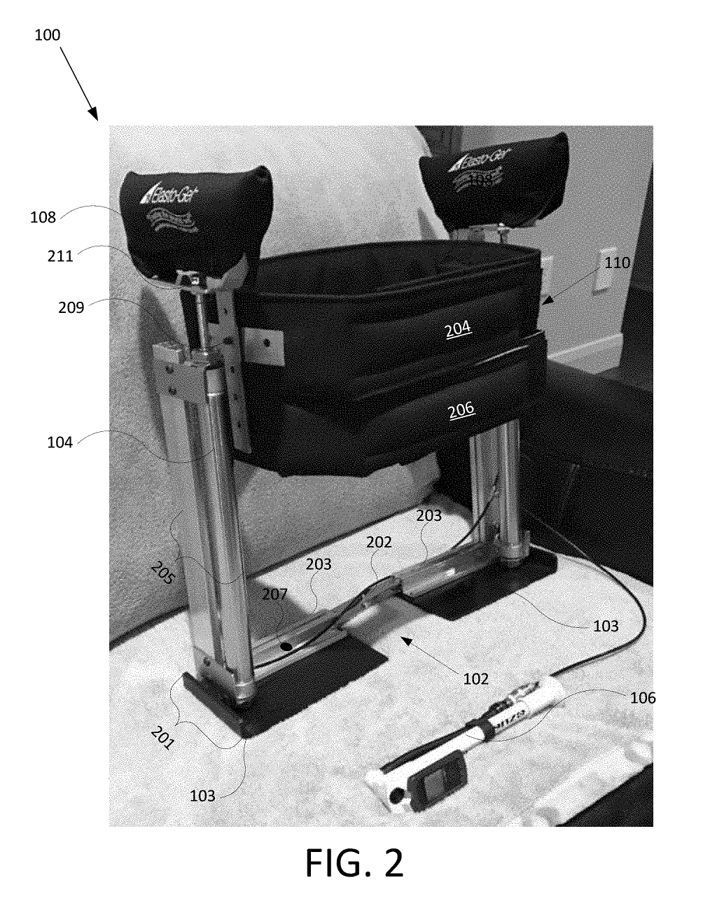

In one embodiment, the base 102 is formed with at least one substantially planar area 103 (see FIG. 2) to evenly distribute the weight of the device 100 and the patient across the seating surface, and thereby not mar the seating surface. In one embodiment, the planar area 102 is provided with a width greater than a width of either the base 102 or upright support 104. The planar area 103 is described below in greater detail with reference to FIG. 2. This beneficially allows a patient to support the upper body above the seating surface while the base 102 distributes the weight of the patient across the seating surface without damaging the seating surface.

In one embodiment, each of the upright supports 104 includes a lifting mechanism 105 capable of lifting the weight of the upper body of the patient. Examples of lifting mechanisms 105 include, but are not limited to infinitely adjustable springs, pneumatic cylinders, hydraulic lifting struts, electric lifting struts, etc. A controller 106 is coupled to the lifting mechanisms 105 and is adapted to increase or decrease the lifting pressure of the lifting mechanisms 105. In one embodiment, the controller 106 may be a manual air or hydraulic pump that when actuated causes the lifting mechanisms to increase a lifting force that raises the underarm supports 108, and subsequently lift a patient off a seating surface. In other embodiments, the controller 106 may be electrical or mechanoelectrical that is configured to actuate pumps and/or motors to extend or retract the arms of the lifting mechanism.

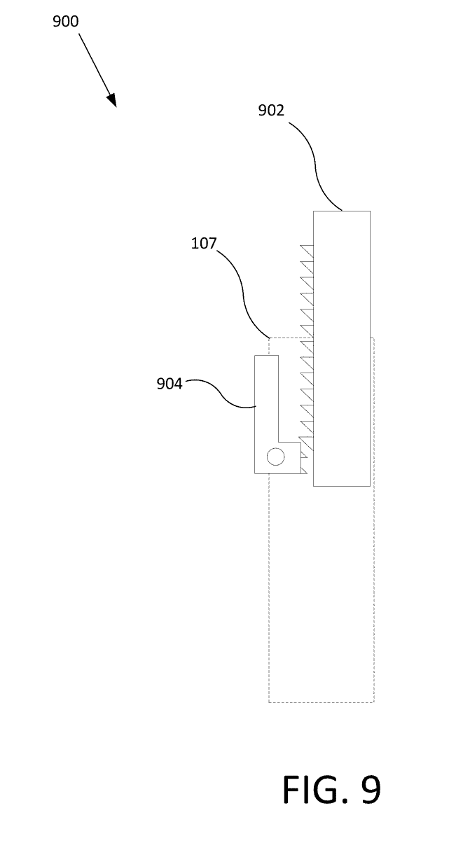

The upright supports 104, in one embodiment, are adjustable and lockable in different positions to allow a patient to adjust the desired height of the underarm supports 108. In the depicted embodiment, the upright support 104 includes a strut 107 extending perpendicularly from the base 102 adjacent to a lifting mechanism 104. The strut 107 may serve as a guide to maintain underarm supports 108 in a correct orientation. Stated differently, the strut 107 works together with the lifting mechanism 105 to maintain an underarm support 108 in a position that comfortably supports the underarm region of a patient. In a further embodiment, the strut 107 may include a locking mechanism, including, but not limited to, a ratcheting mechanism (see FIG. 9), a pin-lock mechanism, etc. In other embodiments, the locking mechanism is incorporated into the lifting mechanism.

As mentioned above, a pair of underarm supports 108 are coupled to the extendable lifting mechanisms. The underarm supports 108, in one embodiment, are padded for the comfort of the patient. The underarm supports 108 may be contoured with a general U or V shape that contours to the underarm area of the patient without affecting blood flow into the patient's arms. The padding, in one embodiment, may be a foam rubber or other suitable material.

In one embodiment, a chest harness 110 is coupled to the underarm supports 108. The coupling, in one embodiment, is adjustable. In other words, the relative position of the chest harness 110 with respect to the underarm supports 108 may be adjusted up or down to accommodate different patients. As such, a coupling point 209 of the chest harness may be raised or lowered with respect to the coupling point 211 of the underarm support. This is depicted in the figures as an “L” bracket with different positioning openings but may be any other adjustable coupling. The chest harness 110, therefore, moves together with the underarm supports 108 in response to the upright supports 104 extending or retracting. In an alternative embodiment, the chest harness 110 is integrally molded with the underarm supports 108. Although not depicted here, an integrally molded unit may resemble a padded vest with built-in underarm supports that are connectable to the upright supports 104. Similarly, the entire device 100 may be incorporated into a chair (e.g., office chair, doctor's chair, or other furniture).

The chest harness 110 is configured to encircle a patient's chest or lower ribcage area. The chest harness 110 may be formed of a semi-rigid material (e.g., planar sheet metal), including but not limited to, a padded and cushioned area with an embedded semi-rigid band or strap 112. The semi-rigid strap 112, in one embodiment, is formed of a flexible metal. The semi-rigid strap 112 strengthens the chest harness 110 so that the chest harness is suitable to at least partially assist in lifting the patient. In other words, the chest harness 110 helps reduce the upper body weight being carried by the underarm supports 108.

This is beneficial because many patients find it uncomfortable and tiring to support their entire weight on underarm supports 108. Advantageously, because the chest harness 110 carries a portion of the upper body weight, the patient retains upper body mobility (i.e., hands and arms are free to move). This unique mobility is accomplished by comfortably supporting the patient's upper body in two areas that do not restrict arm movement (e.g., underarm and chest or lower ribcage). Additionally, semi-flexible materials may be utilized in certain areas of the U-shaped frame (or by using flexible connection joints, or joints that can swivel) to allow the upper body to move freely while being supported or lifted.

In another embodiment, the chest harness 110 may be formed of one or more straps. For example, the one or more straps includes a strap to secure the upper semi-rigid part of a belt to the chest area, and another lower strap to pull the chest harness tight under the lower ribcage. In essence, this creates an hourglass-type shape to help the chest harness contour to the shape of the lower ribcage (see FIG. 2).

FIG. 2 is another perspective view diagram illustrating one embodiment of the device 100 in accordance with embodiments of the disclosure. The device 100, as described above, includes planar areas 103 for distributing the weight of the patient across the seating surface. One or more planar areas or pads 103 may be attached to a bottom surface of the base 102. As described above, the pads 103 may have a width 201 greater than a width 205 of the upright support 104 to provide a stable base for the device 100. The planar areas 103 may have upwardly curving edges to prevent the planar areas 103 from marring the seating surface. In one embodiment, a single planar area 103 spans the entire distance between upright supports 104. Alternatively, two or more planar areas 103 (as depicted in FIG. 2) may be attached to a bottom (i.e., surface that engages the seating surface) of the base 102.

In one embodiment, the base 102 is formed of multiple, sliding components 202, 203. The components may allow the base to extend and retract to accommodate patients of different sizes. A central bar 202 may slidingly engage the bar receivers 203 that are disposed at each end of the base 102. The upright supports 104 may be coupled with the bar receivers 203. In one embodiment, the coupling between the upright supports 104 and the base 102 is flexible and/or capable of rotating/pivoting. This beneficially allows the device to pivot or twist when a patient, for example, reaches for an object. Accordingly, the underarm supports 108 can move radially (i.e., following a radial path with respect to the base) independent of one another, while moving together vertically (with respect to the base). In one embodiment, the coupling between the upright supports 104 and the base 102 are swivel couplings. In other embodiments, the base 102 is capable of a twisting motion that allows each upright support 104 to move radially (i.e., to sweep as the second hand of a clock would). Stated differently, the central bar 202 may be formed of a material that allows for a twisting motion if the upright supports 104 were to move radially in different directions such as when a patient would turn to one side or the other.

The bar receivers 203, in one embodiment, are lockable in different positions with respect to the central bar. In some embodiments, fasteners 207 lock the bar receivers 203 with respect to the bar 202. In alternative embodiment, other locking mechanisms, such as latches, pins, etc., are contemplated. Additionally, the upright supports 104 may be extended and locked in certain positions to accommodate patients of different sizes.

In one embodiment, the chest harness 110, may include a first strap 204 and a second strap 206. The straps 204, 206 may be stitched or otherwise attached to the chest harness 110. The first strap 204 and the second strap 206 allow the chest harness to be fastened around patients of different sizes. As is known, some patients may have a larger lower ribcage than upper ribcage area, and vice versa. Each of the multiple straps 204, 206 are positionable to allow for larger or smaller patients.

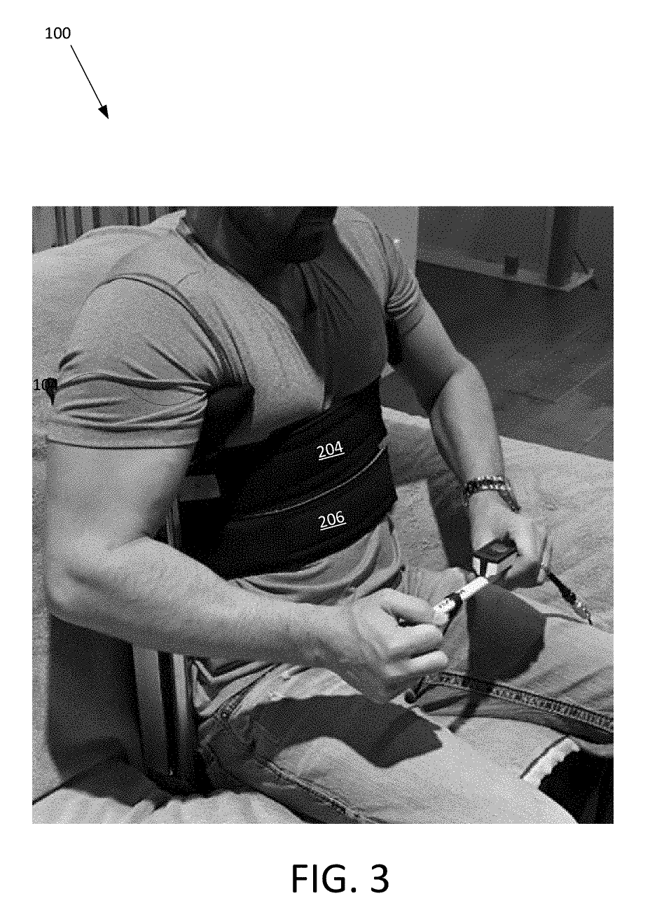

FIG. 3 is a perspective view diagram illustrating one embodiment of a patient using the device 100 in accordance with embodiments of the present disclosure. In one embodiment, the patient sits on or adjacent to the base and engages the chest harness 110. In other words, the patient straps the chest harness around his or her lower or upper ribcage, wherever is more comfortable for the patient. The underarm supports 104 are positioned in the underarm area of the patient. The patient then engages the controller 106 to actuate the lifting mechanisms. In one embodiment, engaging the lifting mechanism comprises manually pumping air into pneumatic struts. Alternatively, electrical or mechanoelectrical mechanisms may be activated to lift the patient.

Once a desired amount of height is reached, to achieve a desired amount of decompression, the controller 106 may lock the position, until otherwise indicated by the patient. In a further embodiment, the patient may desire to completely lift the patient off the seating surface.

As described above, the device 100 is suitable for use on many different seating surfaces. The pads described above with reference to FIG. 3 are configured with rounded edges so as to not mar or otherwise damage the seating surface.

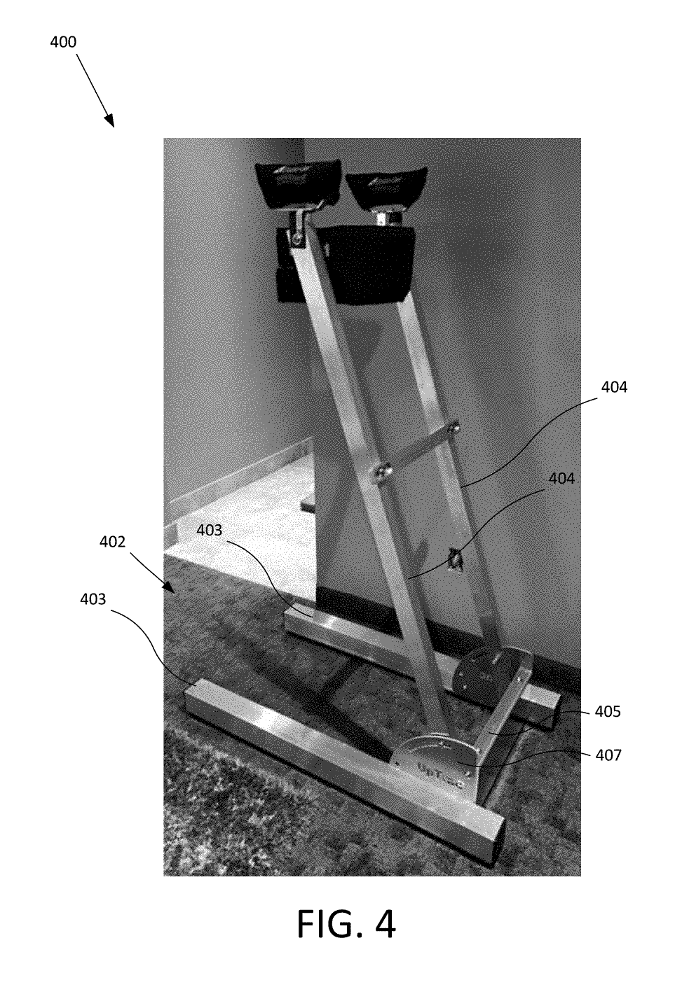

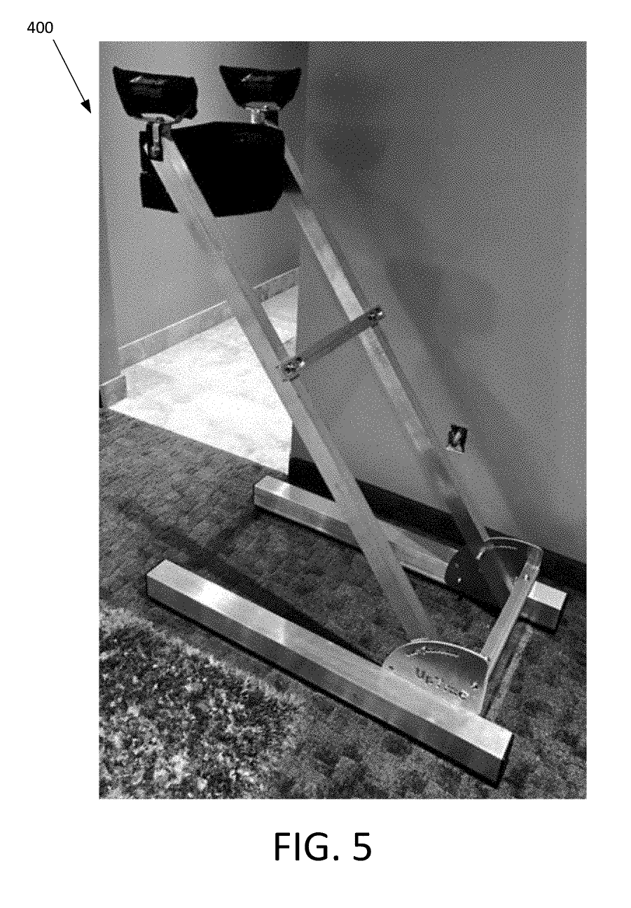

FIGS. 4-8 are different view photographs illustrating one embodiment of a framework that utilizes the chest harness 110 and underarm supports 108 in accordance with embodiments of the present disclosure. In the depicted framework, the patient may strap his or herself into the chest harness 110 and simply relax his or her legs to allow gravity to pull on the lower body for spinal decompression and spinal traction.

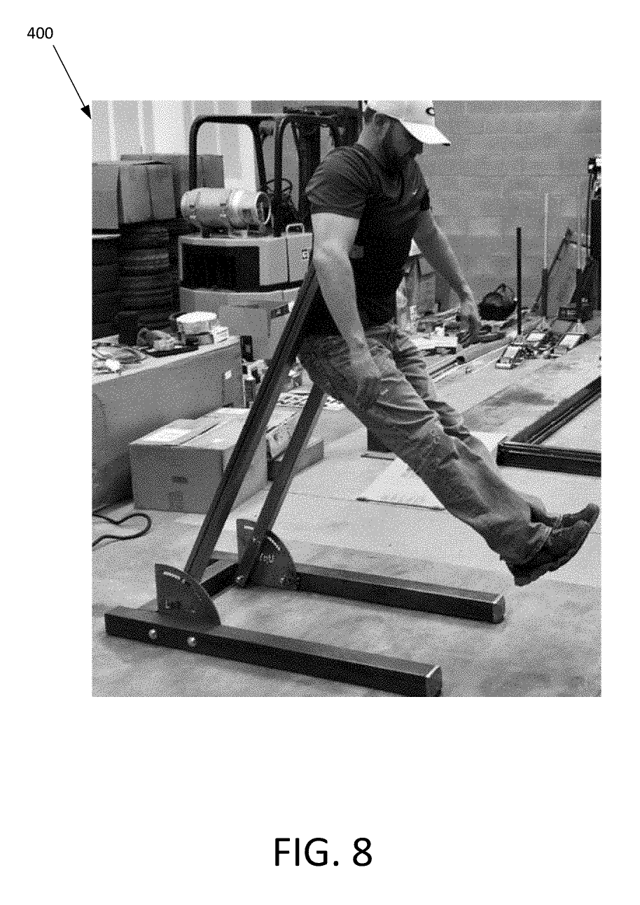

Additionally, a patient may perform exercises in a manner that is similar to exercises that are performed in exercise equipment known as a “Captain's Chairs,” but without the strain of supporting the patient's entire body weight on his or hers elbows and forearms (see FIG. 8).

The framework 400, in one embodiment, includes a base 402 formed of a rigid material. As depicted, the framework 400 may be formed of square tube metal. In alternative embodiments, the framework 400 is formed of any suitable material that is capable of supporting the weight of a patient. The base may be formed with two parallel supports 403 and a center support 405 disposed between the parallel supports 403.

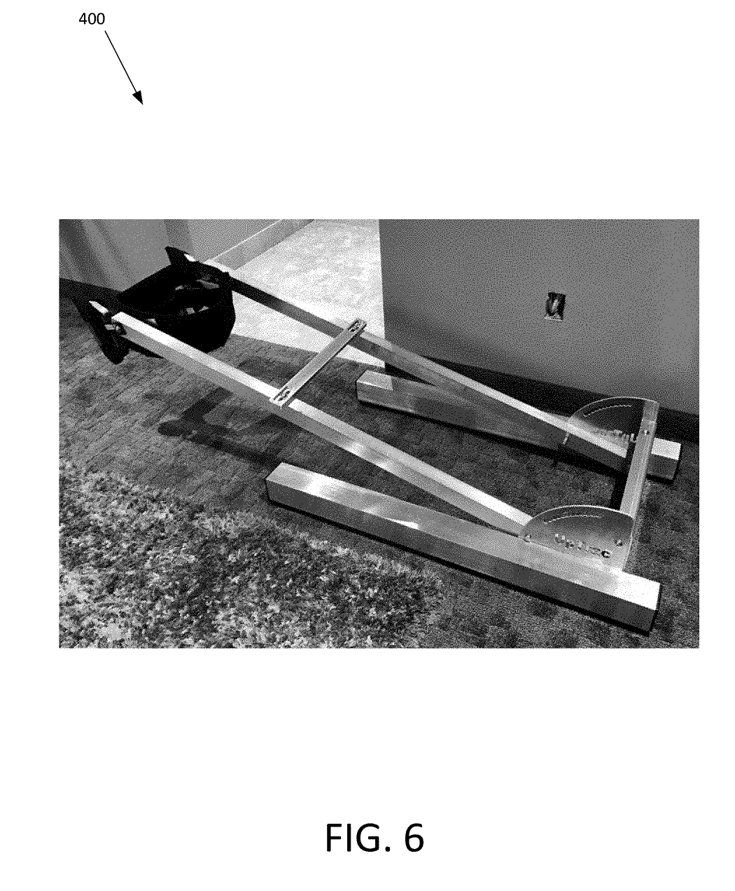

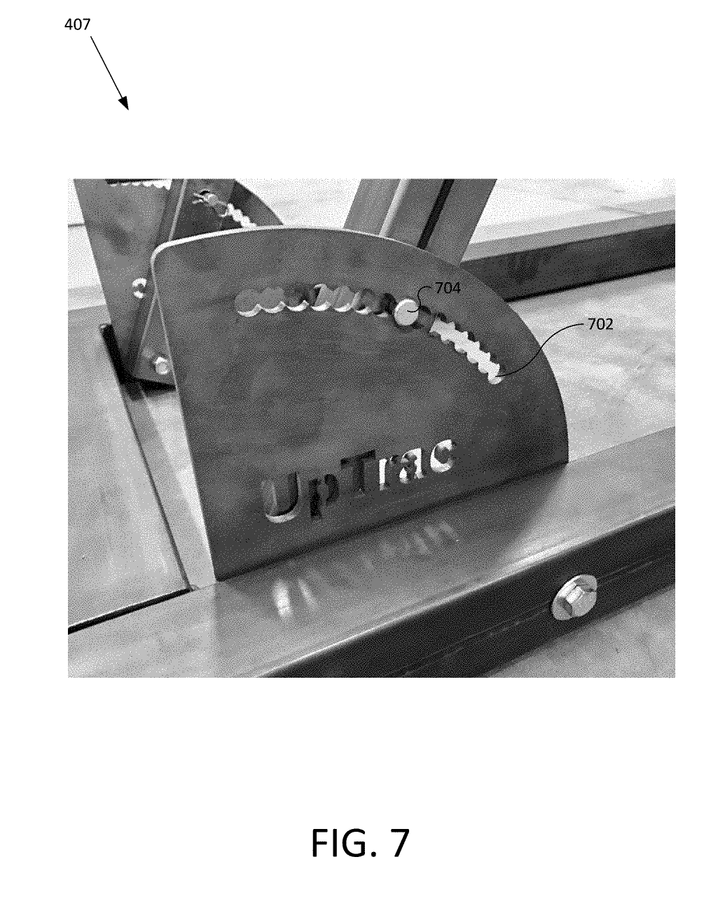

Coupled to the base 402, in one embodiment, are patient support members 404. The patient support members are rotatably coupled to the base 402, and positionable in multiple positions via a positioning brace 407. As depicted, especially in FIG. 7, the positioning brace 407 includes a plurality of locking positions 702 that trace an arc through which a point on the patient support members 404 passes as the patient support members 404 are rotated from an upright position (see FIG. 4) to a collapsed position (see FIG. 6). An opening may be positioned at that point, and a fastener placed through one of the plurality of locking positions and into the opening. Alternatively, a spring-loaded pin 704 may be extendable out of the patient support member 404 and into one of the locking positions 702. To move the patient support members 404, the patient would depress the spring loaded or biased pin and reposition the patient support arms 404.

FIG. 9 is a partial cross-section diagram illustrating one embodiment of a ratcheting mechanism for locking a strut 107 in a desired position in accordance with embodiments of the present disclosure. The ratcheting mechanism 900, in one example, may include the strut 107, an extension rod 902, and a release mechanism 904. The rod 902 may be formed with a plurality of teeth that are configured to engage with teeth of the release mechanism 904. As the lifting mechanism raises the patient, the strut 107 may lock at each tooth automatically to ensure that the patient does not fall unintendedly.

Reference throughout this specification to features, advantages, or similar language does not imply that all of the features and advantages that may be realized with the subject matter of the present disclosure should be or are in any single embodiment. Rather, language referring to the features and advantages is understood to mean that a specific feature, advantage, or characteristic described in connection with an embodiment is included in at least one embodiment of the present disclosure. Thus, discussion of the features and advantages, and similar language, throughout this specification may, but do not necessarily, refer to the same embodiment.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

Furthermore, the details, including the features, structures, or characteristics, of the subject matter described herein may be combined in any suitable manner in one or more embodiments. One skilled in the relevant art will recognize, however, that the subject matter may be practiced without one or more of the specific details, or with other methods, components, materials, and so forth. In other instances, well-known structures, materials, or operations are not shown or described in detail to avoid obscuring aspects of the disclosed subject matter.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A spinal traction device comprising:
an elongated base having a first end and a second end;
a first upright support member coupled to the first end and extending substantially perpendicularly from the base, the first upright support member comprising a first lifting mechanism and a first underarm support;
a second upright support member coupled to the second end and extending substantially perpendicularly from the base, the second upright support member comprising a second lifting mechanism and a second underarm support;
a chest harness configured to encircle a ribcage area of a patient, where the chest harness is coupled on a first side to the first underarm support member and coupled on a second side to the second underarm support member.

2. The apparatus of claim 1, where the elongated base comprises a first receiver, a second receiver, and a central bar disposed between, and slidable into, the first receiver and the second receiver, and configured to adjust a distance between the first upright support member and the second upright support member.

3. The apparatus of claim 2, further comprising a first planar pad coupled to the first receiver, and a second planar pad coupled to the second receiver.

4. The apparatus of claim 3, where each of the first planar pad and the second planar pad comprise at least one curved edge.

5. The apparatus of claim 4, where each of the first planar pad and the second planar pad have a width greater than a width of either the first upright support member or the second upright support member.

6. The apparatus of claim 2, where the central bar is formed of a semi-rigid material.

7. The apparatus of claim 1, further comprising an adjustable bracket disposed between the first underarm support member and the first side of the chest harness, where the adjustable bracket is configured to adjust a distance between a coupling point of the first side of the chest harness and a coupling point of the first underarm support member.

8. The apparatus of claim 1, further comprising an adjustable bracket disposed between the second underarm support member and the second side of the chest harness, where the adjustable bracket is configured to adjust a distance between a coupling point of the second side of the chest harness and a coupling point of the first underarm support member.

9. The apparatus of claim 1, further comprising a controller configured to control the first lifting mechanism and the second lifting mechanism.

10. The apparatus of claim 9, where the first lifting mechanism and the second lifting mechanism are selected from the group consisting of an infinitely adjustable spring, a pneumatic lifting strut, a hydraulic lifting strut, and an electronic lifting strut.

11. The apparatus of claim 10, where the first lifting mechanism and the second lifting mechanism comprise pneumatic lifting struts, and the controller comprises a pump configured to increase or decrease an air pressure in each of the first lifting mechanism and the second lifting mechanism.

12. The apparatus of claim 1, where the chest harness comprises at least one strap having a semi-rigid planar material disposed within a padded strap.

13. The apparatus of claim 12, where the chest harness comprises a pair of independently adjustable straps.

14. A combination system for traction and exercise, the system comprising:
a support base comprising at least a pair of parallel supports and a center support, where the center support is disposed between and couples the parallel supports, the support base configured to engage a floor;
a pair of positioning braces, each of the pair of positioning braces coupled to one of the pair of parallel supports; where each of the pair of positioning braces comprises a plurality of locking positions;
a pair of support arms pivotally coupled to the support base and pivotable between a collapsed position an upright position, where one of the pair of support arms is coupled to one of the pair of parallel supports, and where one of the pair of positions braces is disposed between one of the pair of support arms and one of the pair of parallel supports;
a pair of underarm support members, each of the pair of underarm support members coupled to one of the pair of support arms; and
a chest harness configured to encircle a ribcage area of a patient, where the chest harness is coupled on a first side to one of the pair of underarm support members and is coupled on a second side to the other one of the pair of underarm support members.

15. The system of claim 14, where each of the plurality of support arms comprises a locking pin configured to engage one of the plurality of locking positions.

16. The system of claim 15, where the plurality of locking positions trace an arc along which the locking pin travels when the pair of support arms pivots from the collapsed position to the upright position.

17. The system of claim 14, where the chest harness comprises at least one strap having a semi-rigid planar material disposed within a padded strap.

18. The system of claim 17, where the chest harness comprises a pair of independently adjustable straps.

19. The system of claim 14, further comprising a cross brace coupling the pair of support members.

20. A spinal traction device comprising:
an elongated base having a first end and a second end, where the elongated base comprises a first receiver, a second receiver, and a central bar disposed between, and slidable into, the first receiver and the second receiver, and configured to adjust a distance between the first upright support member and the second upright support member;
a first planar pad coupled to the first receiver, and a second planar pad coupled to the second receiver, where each of the first planar pad and the second planar pad have a width greater than a width of either the first upright support member or the second upright support member;
a first upright support member coupled to the first end and extending substantially perpendicularly from the base, the first upright support member comprising a first lifting mechanism and a first underarm support;
a second upright support member coupled to the second end and extending substantially perpendicularly from the base, the second upright support member comprising a second lifting mechanism and a second underarm support;
a chest harness configured to encircle a ribcage area of a patient, where the chest harness is coupled on a first side to the first underarm support member and coupled on a second side to the second underarm support member.
